(19) United States
(12) Patent Application Publication
(10) Pub. No.: US 2024/0301359 A1
(43) Pub. Date: Sep. 12, 2024

(54) SYSTEM COMPRISING CISPLATIN-RESISTANT XENOGRAFT FOR NASOPHARYNGEAL CARCINOMA AND METHODS THEREOF

(71) Applicant: THE UNIVERSITY OF HONG KONG, Hong Kong (CN)

(72) Inventors: Ho Fun Victor LEE, Hong Kong (CN); Ka Yee LI, Hong Kong (CN)

(73) Assignee: THE UNIVERSITY OF HONG KONG, Hong Kong (CN)

(21) Appl. No.: 18/597,181

(22) Filed: Mar. 6, 2024

Related U.S. Application Data
(60) Provisional application No. 63/488,525, filed on Mar. 6, 2023.

(51) Int. Cl. C12N 5/071; C12N 5/09; G01N 33/50

(52) U.S. Cl. CPC C12N 5/0688; C12N 5/0693; G01N 33/5011; C12N 2503/02; G01N 2500/10

(57) ABSTRACT

The present disclosure relates to a world first and only authentic EBV-positive cisplatin-resistant NPC PDX, xeno76-CR, which has been tested with different examinations and is confirmed as cisplatin-resistant EBV-positive undifferentiated human NPC. Xeno76-CR is a valuable preclinical model that mimics the NPC patients who have failed chemotherapy. It could predict the treatment outcome in drug testings, increase the success rate for new therapeutic agents when proceed into clinical trial, and ultimately benefit NPC patients with more choices of promising therapy.

The present application claims priority to U.S. provisional application Ser. No. 63/488,525 filed Mar. 6, 2023, the contents of which are incorporated by reference its entirety.

### 1. FIELD

The present disclosure generally relates to at least the fields of molecular biology, cell biology, medicine, life sciences research, cancer research, drug models, therapeutic testing. The present disclosure provides patient-derived xenografts (PDX) prepared by transplanting tissue from nasopharyngeal carcinoma (NPC) patients into mice. The present disclosure further provides methods of preparing cisplatin-resistant-PDX and cell lines from the NPC-PDX.

### 2. BACKGROUND

Nasopharyngeal carcinoma (NPC) is a rare cancer worldwide but is the seventh commonest cancer in Hong Kong males in 2020. When compared with other cancer types, such as lung and colorectal cancer, the research progress on NPC is lagging and currently there is no targeted therapy approved for NPC. One of the possible reasons is the research studies on the underlying mechanisms of NPC tumorigenesis and drug development are lacking when compared with other cancer types, and therefore no targeted therapy yet available and approved for NPC patients.

Previous studies attempted to develop cisplatin-resistant NPC models from HNE1 and CNE2 cell lines for drug-resistant pathway evaluation and preclinical drug testing (Jiang et al., 2003; Lin et al., 2008). Abundant drug-resistant pathway-related molecular biology studies have been published based on these cisplatin-resistant NPC models (Guan et al., 2020).

However, the authenticity of most NPC cell lines, including HNE1 and CNE2, is questionable. Both HNE1 and CNE2 were reported EBV negative, and CNE2 was reported contaminated with HeLa cell (Chan et al., 2008). The STR profile of CNE1 and CNE2 were found surprisingly similar, containing at least one identical allele from HeLa and the same specific mutations on p53, even though CNE1 and CNE2 originated around 40 years ago from a well-differentiated squamous carcinoma and poorly differentiated carcinoma NPC tissue respectively (Chan et al., 2008; College, 1978; Gu et al., 1983). It is postulated that CNE1 and CNE2 were hybrids generated by a fusion of HeLa with an unknown NPC cell line. Studies on these misidentified NPC models may lead to a false conclusion.

In view of the disadvantages of the existing technologies, the establishment of a new chemo- or radiotherapy-resistant NPC preclinical model is in demand for addressing the fact that targeted therapy aims for multiple-chemotherapy failed advanced-stage NPC.

### 3. SUMMARY

It is an objective of the present disclosure to provide cisplatin-resistant cell lines and PDXs to solve the aforementioned technical problems. The inventors have successfully established several PDXs by transplanting NPC patient's tissue into mice. Characterization of the PDX are as described in Lin et al. 2018, the contents of which are incorporated by reference in their entirety as if fully set forth herein.

The present disclosure relates to a world first and only authentic EBV-positive cisplatin-resistant NPC PDX, designated herein xeno76-CR, which can bring enormous progress in NPC research, such as understanding in drug resistance pathways and more successful drug development. Xeno76-CR has been tested with different examinations and is confirmed as cisplatin-resistant EBV-positive undifferentiated human NPC. Xeno76-CR is a valuable preclinical model that mimics the NPC patients who have failed chemotherapy. It could predict the treatment outcome in drug testings, increase the success rate for new therapeutic agents when proceed into clinical trial, and ultimately benefit NPC patients with more choices of promising therapy. For example, the present disclosure a targeted drug, ixazomib, on xeno76-CR and found promising results.

In some embodiments, the cisplatin-resistant PDX of the present disclosure can lead to substantial progress and provide a favourable platform in further NPC research, such as identifying potential mutations, targets or signaling pathways take that part in cisplatin-resistance in NPC patients, the potential targets for developing targeted therapies, and providing early prediction on the treatment outcome and prognosis in NPC patients.

In some embodiments, provided is a cisplatin resistant xenograft model for nasopharyngeal carcinoma (NPC) comprising a patient-derived xenograft (PDX) having a short tandem repeat (STR) DNA genomic profile derived from a NPC patient.

In some embodiments, provided is a cisplatin resistant xenograft model for nasopharyngeal carcinoma (NPC) comprising a NPC patient-derived xenograft (PDX) having a short tandem repeat (STR) DNA genomic profile derived from said NPC patient.

In some embodiments, provided is a cisplatin resistant xenograft model for nasopharyngeal cancer comprising a patient-derived xenograft with the same short tandem repeat DNA genomic profile as the original patient.

In some embodiments, provided is a cisplatin resistant xenograft model for nasopharyngeal carcinoma (NPC) comprising a patient-derived xenograft (PDX) having a short tandem repeat (STR) DNA genomic profile derived from a NPC patient.

In some embodiments, provided is a patient-derived xenograft (PDX) from nasopharyngeal carcinoma (NPC) tissue (NPC PDX), wherein the NPC-PDX is resistant to cisplatin (CR).

In some embodiments, the cisplatin-resistant NPC PDX comprises a short tandem repeat (STR) profile of at least one genome DNA as set forth in Table 1. In some embodiments, the at least one genome DNA comprises at least one locus selected from the group consisting of D3S1358, D7S820, vWA, FGA, D8S1179, D21S11, D18S51, D5S818, D13S317, D16S539, TH01, TPOX, CSF1PO, Penta D, and Penta E. In some embodiments, the patient-derived xenograft comprises the same short tandem repeat DNA genomic profile as the original patient.

In some embodiments, the cisplatin-resistant NPC PDX which comprises a short tandem repeat (STR) profile genome DNA substantially as shown in Table 1. In some embodiments, the NPC PDX is EBV1 positive.

In some embodiments, provided a cell line derived from the cisplatin-resistant NPC PDX according to the present disclosure.

In some embodiments, provided is a method of preparing a cisplatin-resistant NPC PDX, the method comprising the steps of: (a) providing a patient-derived xenograft (PDX) from tissue of a nasopharyngeal carcinoma (NPC) patient (NPC PDX), (b) transplanting said NPC PDX into a mouse; and (c) administering to said mice cisplatin at a dose and time sufficient to induce resistance to cisplatin. In some embodiments, the cisplatin is administered at dose of from 1 mg/kg to about 10 mg/kg. In some embodiments, the cisplatin is administered daily for a period of up to 4 weeks. In some embodiments, the mouse is a nude mouse.

In some embodiments, provided is a method of identifying a chemotherapeutic agent capable of treating nasopharyngeal carcinoma (NPC), the method comprising the step of: (a) contacting at least one test chemotherapeutic agent with a cisplatin-resistant NPC PDX according to the present disclosure, and (b) measuring the size of the cisplatin-resistant NPC PDX in the presence and absence of said at least one test chemotherapeutic agent.

In some embodiments, provided is a method of identifying a chemotherapeutic agent capable of treating nasopharyngeal carcinoma (NPC), the method comprising the step of: (a) administering at least one test chemotherapeutic agent to mice comprising a cisplatin-resistant NPC PDX according to the present disclosure, and (b) measuring the size of the cisplatin-resistant NPC PDX in said mice in the presence and absence of said at least one test chemotherapeutic agent.

In some embodiment, the method further comprises the step of comparing the size of the cisplatin-resistant NPC PDX in the presence of the test chemotherapeutic agent to a control NPC PDX not treated with the test chemotherapeutic agent.

Various chemotherapeutic agents can be screened using the methods of the present disclosure. In some embodiments, the chemotherapeutic agent is ixazomib.

In some embodiments, provided is a method of for identifying a test therapeutic agent to treat nasopharyngeal carcinoma (NPC), comprising the steps of a) contacting the test therapeutic agent with a plurality of cells derived from the cisplatin resistant NPC PDX according to the present disclosure; and b) determining the quantify of the cells or the half maximal inhibitory concentration level (IC50) of the test therapeutic agent in the cells.

### 4. BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic diagram of establishment procedure of xeno76-CR;

FIG. 2A depicts Xeno76 and xeno76-CR tumor growth curves of control (2.5% DMF in saline) and treatment (3 mg/kg cisplatin in saline) groups during the three-week treatment period;

FIG. 2B shows images of size and appearance of the xeno76 and xeno76-CR tumor-bearing mice on day 21;

FIG. 2C depicts the tumor weight of xeno76 and xeno76-CR between control and treatment groups;

FIG. 2D depicts the change of body weight of xeno76 and xeno76-CR tumor-bearing mice in control and treatment groups during the treatment period;

FIG. 3A shows EBER staining (20×) results of the paraffin waxed tissue slides of xeno76 and xeno76-CR counter-stained nucleus with methyl green. The scale bar shows a ratio of 1:100 μm;

FIG. 3B depicts EBV copy number per cell in xeno76 and xeno76-CR before and after cisplatin treatment estimated by qPCR;

FIG. 4 shows histological staining (H&E staining and IHC staining) results for xeno76 and xeno76-CR;

FIG. 5A depicts Xeno76-CR tumor volume (mm³) growth curves of control and 4 mg/kg ixazomib treatment groups during the four-week treatment period;

FIG. 5B shows images of size and appearance of the xeno76-CR tumor-bearing mice on day 21;

FIG. 5C depicts tumor weight (g) of xeno76-CR between control and treatment groups; and

FIG. 5D depicts the change of body weight (g) of xeno76-CR bearing mice in control and treatment groups during the treatment period.

### 5. DETAILED DESCRIPTION

In the following description, NPC cell lines and PDXs are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

### 5.1 Cisplatin-Resistant NPC-PDX

Patient-derived xenograft (PDX) is one of the preclinical models widely used in preclinical research, especially in drug development studies. PDX is established by transplanting a patient's tumor tissue into an immunodeficient mouse which mimics the human body environment for the tumor tissue growth and allows new interventional or experimental testing.

In some embodiments, the present disclosure provides a cisplatin-resistant NPC PDX, designated xeno76-CR. In some embodiments, xeno76-CR is characterized by a short tandem repeat (STR) profile having at least one as set forth in Table 1.

Provided is a method of preparing a cisplatin-resistant NPC PDX, the method comprising the steps of: (a) providing a patient-derived xenograft (PDX) from nasopharyngeal carcinoma (NPC) tissue (NPC PDX), (b) transplanting said NPC PDX into a mouse; and (c) administering to said mice cisplatin at a dose and time sufficient to induce resistance to cisplatin.

In some embodiments, the cisplatin is administered at dose of from 1 mg/kg to about 10 mg/kg. In some embodiments, the cisplatin is administered daily for a period of up to 4 weeks, for example about 1 week, about 2 weeks, about 3 weeks, about 4 weeks, or any time in-between. In some embodiments, the mouse is a nude mouse.

In some embodiments, the cisplatin-resistant NPC PDX is prepared by transplanting a NPC PDX into nude mice as described in Lin et a. 2018, and treating the mice with cisplatin until the tumor is unresponsive to the MTD of cisplatin in nude mice. Treatment with cisplatin can be daily, every other day, biweekly, weekly and the like. In some embodiments, cisplatin is administered to the nude mice intravenously. In some embodiments, cisplatin is administered to the nude mice intraperitoneally. In some embodiments, cisplatin is administered to the nude mice subcutaneously. In some embodiments, cisplatin is administered at a dose of about 0.1 mg/kg to about 10 mg/kg, for examples 0.1 mg/kg, 0.5 mg/kg, 1 mg/kg, 2 mg/kg, 1 mg/kg, 3 mg/kg, 4 mg/kg, 5 mg/kg, 5 mg/kg, 7 mg/kg, 8 mg/kg, 9 mg/kg, or 10 mg/kg. In some embodiments, cisplatin is administered for at least about 1 week, 2 weeks, 3 weeks, 4 weeks or longer, until the tumor becomes unresponsive to cisplatin in the mice. The PDX tissue is then harvested from the mice and transplanted into new mice. Cisplatin resistance is then maintained in my mice by weekly administration of cisplatin to maintain resistance.

In some embodiments, cisplatin-resistant NPC PDX is established by treating the xeno76 PDX-bearing nude mice with increasing cisplatin i.p. weekly until the tumor is unresponsive to the MTD of cisplatin in nude mice (5 mg/kg). The xeno76-CR is then subcutaneously maintained in nude mice with weekly 5 mg/kg cisplatin treatment to ensure its resistance to cisplatin.

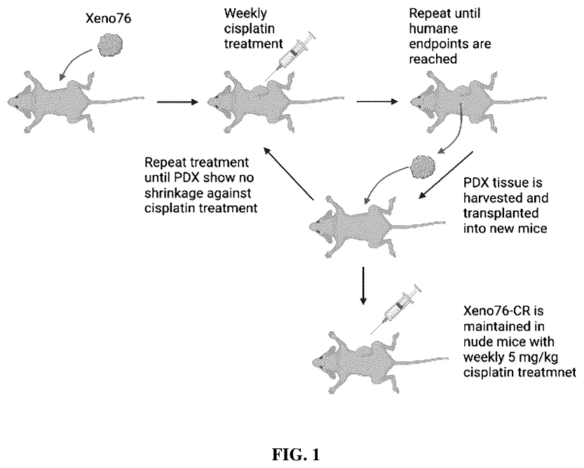

The schematic diagram of establishment procedure of xeno76-CR is shown in FIG. 1.

In some embodiments, the tumor content of xeno76 and xeno76-CR are composed of stromal cells, such as fibroblasts or pericytes. In some embodiments, cells as disclosed herein are co-cultured with immune or other stromal cells.

### 5.2 Screening and Therapeutic Applications

Further in another aspect, the present disclosure discloses a non-human in vitro model and/or in vivo model adapted for evaluating efficacy of an agent against NPC comprising cisplatin-resistant patient-derived NPC xenograft cells.

In some embodiments, the cisplatin-resistant NPC-PDX of the present disclosure can be used in therapy, to identify potential mutations, targets or signaling pathways that form part in cisplatin-resistance in NPC patients. The cisplatin-resistant NPC-PDX of the present disclosure can be potential targets for developing targeted therapies, and providing early prediction on the treatment outcome and prognosis in NPC patients.

For example, in some embodiments, provided is a method of identifying a chemotherapeutic agent capable of treating nasopharyngeal carcinoma (NPC), the method comprising the step of: (a) contacting at least one test chemotherapeutic agent with a cisplatin-resistant NPC PDX according to the present disclosure, and (b) measuring the size of the cisplatin-resistant NPC PDX in the presence and absence of said at least one test chemotherapeutic agent.

In some embodiments, provided is a method of identifying a chemotherapeutic agent capable of treating nasopharyngeal carcinoma (NPC), the method comprising the step of: (a) administering at least one test chemotherapeutic agent to mice comprising a cisplatin-resistant NPC PDX according to the present disclosure, and (b) measuring the size of the cisplatin-resistant NPC PDX in said mice in the presence and absence of said at least one test chemotherapeutic agent.

In some embodiment, the method further comprises the step of comparing the size of the cisplatin-resistant NPC PDX in the presence of the test chemotherapeutic agent to a control NPC PDX not treated with the test chemotherapeutic agent.

Various chemotherapeutic agents can be screened using the methods of the present disclosure. In some embodiments, the chemotherapeutic agent is ixazomib. Other examples of chemotherapeutic agents include etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

In some embodiments, provided is a method of for identifying a test therapeutic agent to treat nasopharyngeal carcinoma (NPC), comprising the steps of a) contacting the test therapeutic agent with a plurality of cells derived from the cisplatin resistant NPC PDX according to the present disclosure; and b) determining the quantify of the cells or the half maximal inhibitory concentration level (IC50) of the test therapeutic agent in the cells.

In some embodiments, provided is a method of for identifying a test therapeutic agent to treat NPC comprising the steps of: (a) contacting a cell derived from the cisplatin-resistant NPC PDX according to the present disclosure with a test chemotherapeutic agent, and measuring the ability of the test chemotherapeutic agent to inhibit growth of said cell.

In a further aspect, the present disclosure provides a method of evaluating the efficacy of an agent used to treat nasopharyngeal carcinoma (NPC) comprising the isolation of NPC cells from NPC patients; implanting/introducing said NPC cells in a non-human model, harvesting tumour xenografts and subjecting said xenografts to a tissue dissociation and digestion process to obtain single cells, and labelling said cells with fluorescent and/or luminescent protein i.e. gfp-luc prior to preparing/adapting the cells for use in further studies in carcinoma. In one embodiment, the isolated NPC cells may be characterised to identify its properties. In one embodiment, the cells may include proven Epstein-Barr virus (EBV) positive properties, and/or cells exhibiting EBV negative properties.

In another aspect, the present disclosure provides a luciferase-based assay to analyse the patient-derived xenografts (PDX) that expresses a fluorescent protein or a luciferase, or a combination thereof, for use in evaluating therapies comprising NPC cells.

In a further aspect, the present disclosure discloses, and with the support of experimental examples, a method of analysing the proliferation of xenograft cells, the method being advantageously sensitive and can specifically measure the real-time proliferation of xenograft cells both in vitro and in vivo. It is anticipated that the method of analysing the proliferation can measure the xenograft cells growth enhancement resulting from the addition of growth supplements as well as from the effect of co-culturing with other human cell types, in addition to the ability to gauge the inhibition of cell viability. It is understood that the inhibition of cell viability may be carried out by other standard means of treatments or new agents to achieve the same purpose.

### 6. EXAMPLES

### 6.1 Example 1: Development of Cisplatin-Resistant NPC PDX Model

A protocol for developing cisplatin-resistant PDX model from the NPC PDX models is described. NPC PDX tissues were implanted in nude mice subcutaneously (FIG. 1). Gradual increase in concentration of cisplatin diluted with saline was then injected to the nude mice weekly intraperitoneally when the PDX tissue has grown 1 cm³ in volume. The tumor volume of each mouse were monitored weekly to determine if higher or lower dose should be given, until the tumor was unresponsive to the maximum tolerated dose of cisplatin in nude mice (5 mg/kg). The xeno76-CR was then maintained in nude mice with regular cisplatin treatment to ensure its resistance to cisplatin.

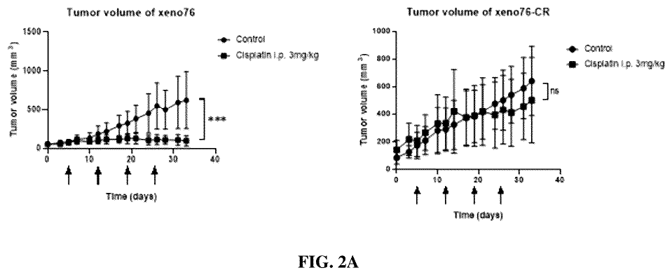

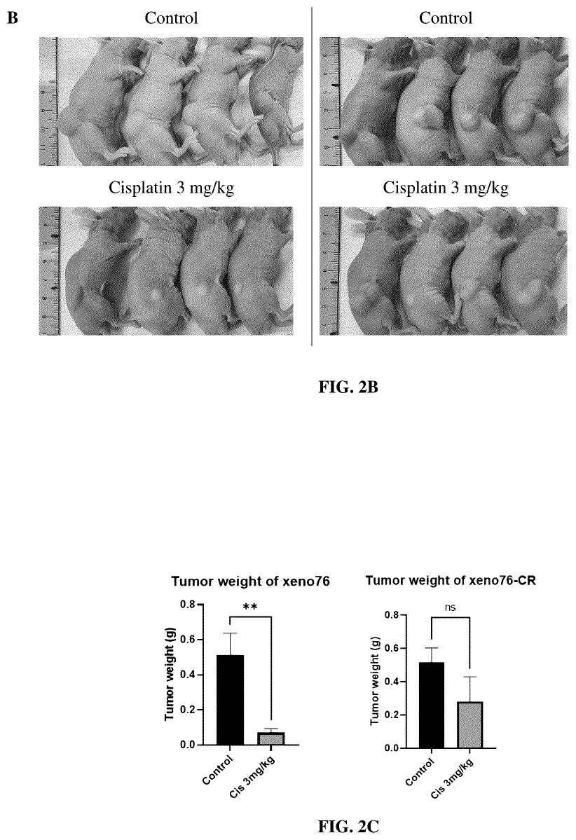

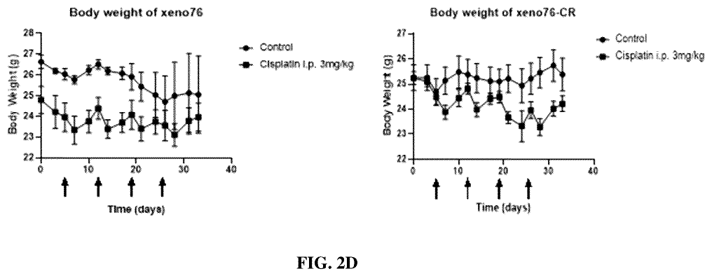

In accordance with one embodiment of the present disclosure, the resistance level to cisplatin in xeno76-CR was first tested and confirmed in-vivo. Parental xeno76 and xeno76-CR-bearing nude mice were randomized into control and treatment groups (n=4). Mice in the control group received the vehicle (2.5% DMF) in saline, while the treatment group was treated with 3 mg/kg cisplatin diluted in saline weekly via i.p. injection for four weeks. The tumor volume was measured and recorded during the whole treatment period, and the tumor growth curves of xeno76 and xeno76-CR are shown in Error! Reference source not found.A. The results in FIG. 2A show the Xeno76 and xeno76-CR tumor growth curves of control (2.5% DMF in saline) and treatment (3 mg/kg cisplatin in saline) groups during the three-week treatment period. Black arrows represent the treatment day. The xeno76 and xeno76-CR nude mice were randomized respectively on day 0 and received the first treatment. Data presented are mean (n=4) with error bars showing SEM. Statistical significance was done using paired two-tailed t-test, *** P<0.001, ns P>0.05.

The images of size and appearance of the xeno76 and xeno76-CR tumor-bearing mice on day 21 are shown in FIG. 2B. The results showed that the growth of xeno76 was significantly inhibited by cisplatin, while the xeno76-CR treatment group showed a similar growth rate to the control group with no significant difference.

Furthermore, the tumors of xeno76 and xeno76-CR were also weighed and harvested for further investigation (FIG. 2C). Statistical significance was done using the unpaired two-tailed t-test, ** P<0.01, ns P>0.05. The tumor weight in cisplatin-treated xeno76 also showed significantly lighter than the xeno76 control group (** P<0.001). In contrast, the control and treatment groups of xeno76-CR have no significant differences, consistent with the findings observed from tumor volume.

Referring to FIG. 2D, the change of body weight of xeno76 and xeno76-CR tumor-bearing mice in control and treatment groups during the treatment period was measured. Data presented are means (n=4) with error bars showing the SEM. Black arrows represent the treatment day. The results showed that the body weight of the xeno76 and xeno76-CR bearing nude mice during the treatment period displayed that the 3 mg/kg cisplatin treatment did not induce body weight loss or any observable toxicities, which suggests the tumor growth inhibition observed in xeno76 treatment group was not affected by the mice conditions. This confirmed that xeno76-CR is resistant to at least 3 mg/kg cisplatin, while the parental xeno76 is sensitive to cisplatin.

To confirm the authenticity and identity of xeno76-CR, the genome DNA of xeno76 at passage 28 and xeno76-CR at passage nine were extracted and sent for short tandem repeat (STR) profiling. STR DNA profiling is a method often used to compare the identities between different DNA samples and detect any cross-contamination between human cell lines by evaluating the number of STR at specific loci of the genome. According to the ANSI/ATCC ASN-0002 Standard of the use of STR profiling for the purpose of cell line authentication testing, the genotypes of 15 loci (D3S1358, D7S820, vWA, FGA, D8S1179, D21S11, D18S51, D5S818, D13S317, D16S539, TH01, TPOX, CSF1PO, Penta D, and Penta E) and Amelogenin at the sex chromosomes were examined. The STR profiles of the blood sample from patient 76 (Lin et al., 2018), xeno76, and xeno76-CR are shown Table. When comparing the STR profile of the blood sample of patient 76 with xeno76, loss of heterozygosity (LOH) at D16S539, D3S1358, D21S11, and Penta D was observed in xeno76-CR. Importantly, xeno76-CR conserved the STR profile from xeno76. This confirmed xeno76-CR is derived from xeno76 with no contamination from other human cell lines or PDXs, and no additional LOH developed, indicating the resistance was not developed by LOH.

TABLE 1
STR profile of genome DNA from patient
76, xeno76, and xeno76-CR

| SMP1 | Patient 76 | Xeno76 P28 | Xeno76-CR P9 |
|---|---|---|---|
| D3S1358 | 16, 17 | 17 | 17 |
| D7S820 | 9, 11 | 9, 11 | 9, 11 |
| vWA | 14, 17 | 14, 17 | 14, 17 |
| FGA | 19, 25 | 19, 25 | 19, 25 |
| D8S1179 | 10, 11 | 10, 11 | 10, 11 |
| D21S11 | 30, 33.2 | 33.2 | 33.2 |
| D18S51 | 13, 15 | 13, 15 | 13, 15 |
| D5S818 | 11, 13 | 11, 13 | 11, 13 |
| D13S317 | 10, 11 | 10, 11 | 10, 11 |
| D16S539 | 10, 11 | 11 | 11 |
| TH01 | 7, 9 | 7, 9 | 7, 9 |
| TPOX | 8, 12 | 8, 12 | 8, 12 |
| CSF1PO | 12, 13 | 12, 13 | 12, 13 |
| AMEL | X | X | X |
| Penta D | 7, 9 | 9 | 9 |
| Penta E | 5, 13 | 5, 13 | 5, 13 |

*SMP smallest main peak

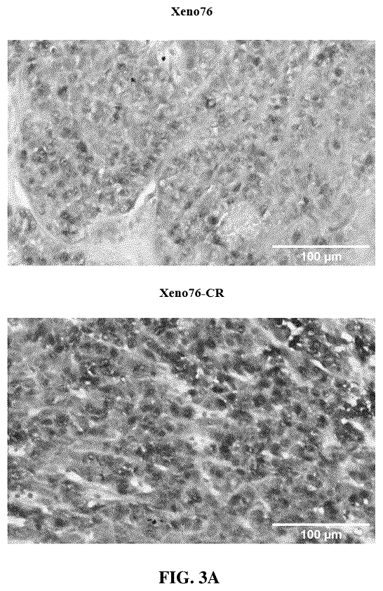

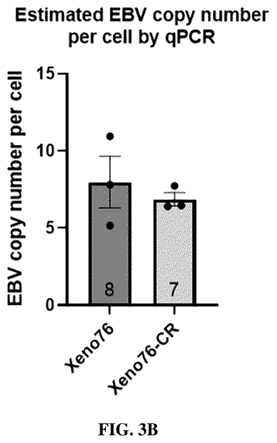

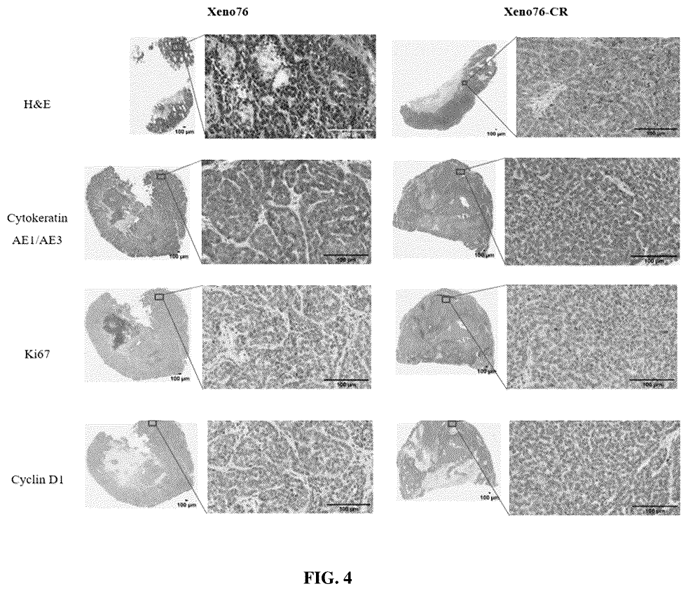

EBV infection plays a critical role in NPC development and chemo-resistance, so the EBV status was next evaluated. EBER staining was performed on xeno76, and xeno76-CR paraffin waxed slides, and the stained slides were scanned by Vectra Polaris automated quantitative pathology imaging system. In accordance with one embodiment of the present disclosure, FIG. 3A shows EBV infection status in xeno76 and xeno76-CRA. The EBER-stained images confirmed that both xeno76 and xeno76-CR are LBV-positive. Moreover, the EBV copy number per cell in xeno76 and xeno76-CR are similar, around 8 and 7, respectively (Error! Reference source not found.B), indicating that the level of EBV copy number per cell may not contribute to the cisplatin resistance pathway. Data in FIG. 3B presented the mean from n=3 independent tumor samples with error bars showing the SEM. Statistical significance was done by unpaired two-tailed t-test, ns P>0.05.

Histology examinations on H&E and IHC stained xeno76 and xeno76-CR paraffin waxed tissue slides were performed to confirm the pathological identity of the PDXs. The stained slides were also scanned, imaged, and quantified by Vectra Polaris's automated quantitative pathology imaging system, as displayed in Error! Reference source not found.

H&E staining results verified the presence of undifferentiated carcinoma cells in both xeno76 and xeno76-CR. The tumor contents in both xeno76 and xeno76-CR are composed of stromal cells, such as fibroblasts. IHC was performed against cytokeratin AE1/AE3, Ki67, and cyclin D1, authenticating that xeno76 and xeno76-CR are both NPC tumor cells with epithelial. The IHC staining against cytokeratin AE1/AE3, Ki67, and cyclin D1 confirmed the epithelial nature of these tumors. Images are shown in a scale bar of 1:100 □m. Xeno76 and xeno76-CR are the first and only pair of authentical EBV positive cisplatin-sensitive and cisplatin-resistant preclinical NPC PDX models for research mimicking NPC patients with chemotherapy-resistant.

In summary, the above results evidenced that xeno76-CR is an authentic EBV-positive cisplatin-resistant NPC PDX. This novel cisplatin-resistant NPC model mimics the NPC patients who have failed cisplatin chemotherapy, the most commonly used chemotherapeutic agent in treating NPC patients. Xeno76-CR can act as a representative model to predict the treatment outcome in drug screening studies, identify drug targets against late-stage NPC patients, evaluate NPC chemo-resistance pathway and foster further NPC cancer research. This The described model can increase the success rate for identifying new therapeutic agents, improve the understanding of NPC and how NPC develops chemo-resistance, and ultimately benefit NPC patients with more choices of promising therapy.

### 6.2 Example 2: Inhibition of Xeno 76-CR Grown with Ixazomib

A preclinical study of ixazomib was then performed on xeno76-CR to evaluate the tumor growth inhibition effect on the cisplatin-resistant model and if xeno76-CR is resistant specifically to cisplatin.

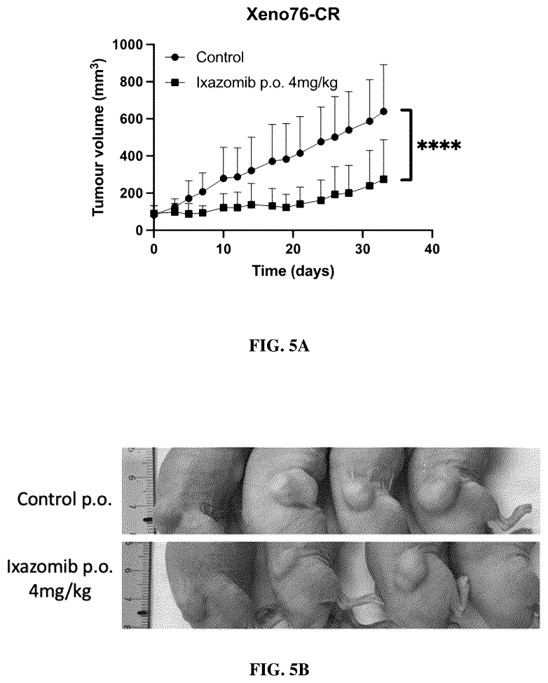

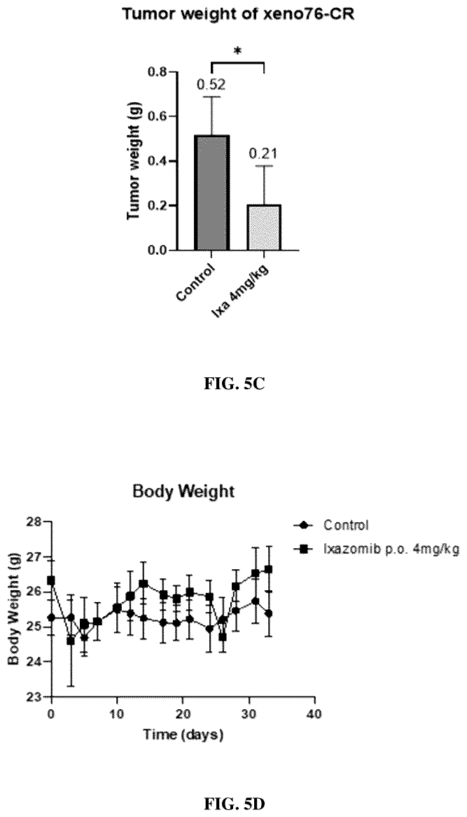

Xeno76-CR-bearing nude mice were randomized into the control and 4 mg/kg ixazomib treatment group (n=4). Referring to FIG. 5A, black arrows represent the treatment day. Data presented are mean (n=4) with error bars showing SEM. Statistical significance was done using one-way ANOVA, **** P<0.0001. The tumor growth curve of xeno76-CR during the three-week treatment, displayed in FIG. 5A, showed that 4 mg/kg of ixazomib effectively inhibited xeno76-CR growth with statistical significance **** P<0.0001.

The images of size and appearance of the xeno76-CR tumor-bearing mice between control and treatment groups on day 21 are shown in FIG. 5B. The tumor weight recorded at the endpoint of the experiment in the treatment group (0.21 g) is significantly smaller than in the control group (0.52 g) (* P<0.05) (FIG. 5C), which is consistent with the reduction of tumor volume observed and further confirmed the tumor inhibition effect in xeno76-CR. This implies that xeno76-CR is resistant specifically to cisplatin but not to general therapeutic agents resistant. Consistent with the previous experiments that 4 mg/kg ixazomib did not induce any observable side effects or weight loss in nude mice (FIG. 5D). In FIG. 5C and FIG. 5D, data presented are means (n=4) with error bars showing the SEM. Statistical significance was done using the unpaired two-tailed t-test. Black arrows represent the treatment day.

In summary, ixazomib inhibited both CDX and PDX which retained the tumor heterogeneity properties and the genetic background with the original patient tumor. Apart from primary NPC (xeno76), which is naïve to any radio- and chemotherapy, metastatic (xeno113) and cisplatin-resistant (xeno76-CR) NPC are all sensitive to ixazomib in a relatively low dosage when compared with other tumor types. Moreover, all the tested animals observed no side effects and body weight loss, revealing that ixazomib is a potentially less toxic cancer drug for advanced-stage NPC patients.

### Exemplary Products, Systems and Methods are Set Out in the Following Embodiments:

1. A cisplatin resistant xenograft model for nasopharyngeal carcinoma (NPC) comprising a patient-derived xenograft (PDX) having a short tandem repeat (STR) DNA genomic profile derived from a NPC patient.
2. A cisplatin resistant xenograft model for nasopharyngeal carcinoma (NPC) comprising a NPC patient-derived xenograft (PDX) having a short tandem repeat (STR) DNA genomic profile derived from said NPC patient.
3. A cisplatin resistant xenograft model for nasopharyngeal cancer comprising a patient-derived xenograft with the same short tandem repeat DNA genomic profile as the original patient.
4. A patient-derived xenograft model (PDX) from nasopharyngeal carcinoma (NPC) tissue (NPC PDX), wherein the NPC PDX is resistant to cisplatin (CR).
5. The cisplatin-resistant NPC PDX according to any one of embodiments 1-4, which comprises a short tandem repeat (STR) profile of at least one genome DNA as set forth in Table 1.
6. The cisplatin-resistant NPC PDX according to embodiment 5, wherein the at least one genome DNA comprises at least one locus selected from the group consisting of D3S1358, D7S820, vWA, FGA, D8S1179, D21S11, D18S51, D5S818, D13S317, D16S539, TH01, TPOX, CSF1PO, Penta D, and Penta E.
7. The cisplatin-resistant NPC PDX according to any one of embodiments 1-6, which comprises a short tandem repeat (STR) profile genome DNA substantially as shown in Table 1.
8. The cisplatin-resistant NPC PDX according to any one of embodiments 1-7, wherein the NPC PDX is EBV1 positive.
9. A cell line comprising the cisplatin-resistant NPC PDX according to any one of embodiments 1-8.
10. A method of preparing the cisplatin-resistant NPC PDX of any one of embodiments 1-9, the method comprising the steps of:
   a. providing a xenograft (PDX) derived from a nasopharyngeal carcinoma (NPC) patient (NPC PDX),
   b. transplanting said NPC PDX into a mouse; and
   c. administering to said mice cisplatin at a dose and time sufficient to induce resistance to cisplatin.
11. The method of embodiment 10, wherein the NPC-PDX is derived from a NPC patient tumor tissue.
12. The method of embodiment 10 or 11 wherein the cisplatin is administered at dose of from 1 mg/kg to about 10 mg/kg.
13. The method of any one of embodiments 10-12, wherein the cisplatin is administered daily for a period of up to 4 weeks.
14. The method of any one of embodiments 10-13, wherein the mouse is a nude mouse.
15. A method of identifying a chemotherapeutic agent capable of treating nasopharyngeal carcinoma (NPC), the method comprising the step of:
   a. contacting at least one test chemotherapeutic agent with a cisplatin-resistant NPC PDX according to any one of embodiments 1-9; and
   b. measuring the size of the cisplatin-resistant NPC PDX in the presence and absence of said at least one test chemotherapeutic agent.
16. A method of identifying a chemotherapeutic agent capable of treating nasopharyngeal carcinoma (NPC), the method comprising the step of:
   a. administering at least one test chemotherapeutic agent to mice comprising a cisplatin-resistant NPC PDX according to any one of embodiments 1-9; and
   b. measuring the size of the cisplatin-resistant NPC PDX in said mice in the presence and absence of said at least one test chemotherapeutic agent.
17. The method according to embodiment 15 or 16, further comprising the step of comparing the size of the cisplatin-resistant NPC PDX in the presence of the test chemotherapeutic agent to a control NPC PDX not treated with the test chemotherapeutic agent.
18. A method of treating a patient having nasopharyngeal carcinoma (NPC) comprising administering to a patient in need thereof a chemotherapeutic agent identified by the method of any one of embodiments 15-17.
19. A patient-derived xenograft (PDX) from nasopharyngeal carcinoma (NPC) tissue (NPC PDX), wherein the NPC-PDX is resistant to a chemotherapeutic agent.
20. The cisplatin-resistant NPC PDX according to embodiment 19, wherein the chemotherapeutic agent is cisplatin.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

### REFERENCES

Chan, S. Y., Choy, K. W., Tsao, S. W., Tao, Q., Tang, T., Chung, G. T., & Lo, K. W. (2008). Authentication of nasopharyngeal carcinoma tumor lines. Int J Cancer, 122(9), 2169-2171. https://doi.org/10.1002/ijc.23374
College, C. A. o. M. S. a. C. M. (1978). Establishment of an epithelioid cell line and a fusiform cell line from a patient with nasopharyngeal carcinoma. Scientia Sinica, 21(1), 127-138.
Gu, S., Tang, W., Zeng, Y., Zhao, M., Zhao, E., Deng, W., & Li, K. (1983). An epithelial cell line established from poorly differentiated nasopharyngeal carcinoma. Chinese Journal of Cancer(2), 70-72.
Guan, S., Wei, J., Huang, L., & Wu, L. (2020). Chemotherapy and chemo-resistance in nasopharyngeal carcinoma. European journal of medicinal chemistry, 207, 112758-112758. https://doi.org/10.1016/j.ejmech.2020.112758
Jiang, R. D., Zhang, L. X., Yue, W., Zhu, Y. F., Lu, H. J., Liu, X., Cen, X. T., Guan, X. Y., & Li, C. H. (2003). [Establishment of a human nasopharyngeal carcinoma drug-resistant cell line CNE2/DDP and screening of drug-resistant genes]. Ai Zheng, 22(4), 337-345.
Lin, W., Li, D., Lin, Y., Du, C., Chen, J., & Hong, C. (2008). Establishment of a cisplatin-induced human nasopharyngeal carcinoma drug-resistant cell line and its biological characteristics. Lin chuang er bi yan hou ke za zhi, 22(21), 992-997.
Lin, W., Yip, Y. L., Jia, L., Deng, W., Zheng, H., Dai, W., Ko, J. M. Y., Lo, K. W., Chung, G. T. Y., Yip, K. Y., Lee, S. D., Kwan, J. S., Zhang, J., Liu, T., Chan, J. Y., Kwong, D. L., Lee, V. H., Nicholls, J. M., Busson, P., . . . Tsao, S. W. (2018). Establishment and characterization of new tumor xenografts and cancer cell lines from EBV-positive nasopharyngeal carcinoma. Nat Commun, 9(1), 4663. https://doi.org/10.1038/s41467-018-06889-5

What is claimed:

1. A cisplatin resistant xenograft model for nasopharyngeal carcinoma (NPC) comprising a patient-derived xenograft (PDX) having a short tandem repeat (STR) DNA genomic profile derived from a NPC patient.

2. The cisplatin-resistant NPC PDX according to claim 1, which comprises a STR profile of at least one genome DNA locus as set forth in Table 1.

3. The cisplatin-resistant NPC PDX according to claim 2, wherein the at least one genome DNA comprises at least one locus selected from the group consisting of D3S1358, D7S820, vWA, FGA, D8S1179, D21S11, D18S51, D5S818, D13S317, D16S539, TH01, TPOX, CSF1PO, Penta D, and Penta E.

4. The cisplatin-resistant NPC PDX according to claim 1, which comprises a short tandem repeat (STR) profile genome DNA substantially as shown in Table 1.

5. The cisplatin-resistant NPC PDX according to claim 1, wherein the NPC PDX is EBV1 positive.

6. A cell line derived from the cisplatin-resistant NPC PDX according to claim 1.

7. A method of preparing the cisplatin-resistant NPC PDX, the method comprising the steps of:
a. providing a xenograft (PDX) derived a from nasopharyngeal carcinoma (NPC) patient (NPC PDX),
b. transplanting said NPC PDX into a mouse; and
c. administering to said mice cisplatin at a dose and time sufficient to induce resistance to cisplatin.

8. The method of claim 7, wherein the NPC-PDX is derived from a NPC patient tumor tissue.

9. The method of claim 7, wherein the cisplatin is administered at dose of from 1 mg/kg to about 10 mg/kg.

10. The method of claim 7, wherein the cisplatin is administered daily for a period of up to 4 weeks.

11. The method of claim 7, wherein the mouse is a nude mouse.

12. A method of identifying a chemotherapeutic agent capable of treating nasopharyngeal carcinoma (NPC), the method comprising the step of:
a. contacting at least one test chemotherapeutic agent with a cisplatin-resistant NPC PDX according to claim 1, and
b. measuring the size of the cisplatin-resistant NPC PDX in the presence and absence of said at least one test chemotherapeutic agent.

13. A method of identifying a chemotherapeutic agent capable of treating nasopharyngeal carcinoma (NPC), the method comprising the step of:
a. administering at least one test chemotherapeutic agent to mice comprising a cisplatin-resistant NPC PDX according to claim 1, and
b. measuring the size of the cisplatin-resistant NPC PDX in said mice in the presence and absence of said at least one test chemotherapeutic agent.

14. The method according to claim 12, further comprising the step of comparing the size of the cisplatin-resistant NPC PDX in the presence of the test chemotherapeutic agent to a control NPC PDX not treated with the test chemotherapeutic agent.

15. A method of identifying a chemotherapeutic agent capable of treating nasopharyngeal carcinoma (NPC), the method comprising the step of:
a. contacting a cell derived from the cisplatin-resistant NPC PDX according to claim 1, with a test chemotherapeutic agent, and
b. measuring the ability of the test chemotherapeutic agent to inhibit growth of said cell.

16. A method of treating a patient having nasopharyngeal carcinoma (NPC) comprising administering to a patient in need thereof a chemotherapeutic agent identified by the method of claim 12.
